(19) United States
(12) Patent Application Publication
(10) Pub. No.: US 2024/0156490 A1
(43) Pub. Date: May 16, 2024

(54) APPARATUS FOR PLACING A NEEDLE AT A SPECIFIC LOCATION AND DEPTH USING AN ULTRASOUND PROBE

(71) Applicant: University of Kentucky Research Foundation, Lexington, KY (US)

(72) Inventor: Kyle Murphy, Lexington, KY (US)

(21) Appl. No.: 18/281,743

(22) PCT Filed: Mar. 22, 2022
(86) PCT No.: PCT/US22/21373

§ 371 Date: Sep. 12, 2023

Related U.S. Application Data
(60) Provisional application No. 63/164,211, filed on Mar. 22, 2021.

(51) Int. Cl. A61B 17/34; A61B 8/00

(52) U.S. Cl. CPC A61B 17/3403; A61B 8/4455; A61B 2017/3411; A61B 2017/3413; A61M 5/427

(57) ABSTRACT

An apparatus adapted for placing a needle at a specific location and depth using an ultrasound probe includes a needle guide with a plurality of needle guide apertures, a support, a stop locator, a syringe, a needle with one or more stops and a probe, such as an ultrasonic probe. The needle guide apertures may be oriented in a first row extending along a length of the needle guide.

### RELATED APPLICATION

This application claims the benefit of U.S. Provisional Patent Application Ser. No. 63/164,211, filed on Mar. 22, 2021, and hereby incorporated by reference in its entirety.

### TECHNICAL FIELD

This document relates to an apparatus adapted for placing a needle at a specific location and depth using an ultrasound probe.

### BACKGROUND

This document relates to a new and improved apparatus specifically adapted to allow one to easily and precisely place a needle at a specific location and a specific depth using an ultrasound probe. In this way it is possible for one to consistently find and place the end or tip of the needle at a desired location, such as in a vessel of a patient or in an abscess cavity in a relatively quick and efficient manner.

### SUMMARY

In accordance with the purposes and benefits described herein, an apparatus is provided for the positioning of a needle at a desired location and depth under the skin of a patient. That apparatus comprises, consists of or consists essentially of a needle guide including a plurality of needle guide apertures. The needle guide apertures may be oriented in a first row extending along a length of the needle guide.

In one or more of the many possible embodiments of the apparatus, the needle guide includes a first section and a second section interconnected along an interface extending along a longitudinal axis of the needle guide and through each aperture of the plurality of needle guide apertures.

In one or more embodiments, the apparatus includes needle guide apertures also oriented in a second row extending parallel to the first row. Still further, the apparatus may also include needle guide apertures oriented in a third row extending parallel to the first row and the second row. Those needle guide apertures may be oriented at an angle of between about 1°-89° (more commonly between about 30°-60°) relative to a longitudinal axis of the needle guide.

In one or more of the many possible embodiments of the apparatus, the apparatus may also include needle depth graduations along a face of the needle guide. Still further, the apparatus may include a stop locator adapted for positioning a plurality of stops on a needle to be guided by the needle guide. That stop locator may include depth graduations and a plurality of stop receivers at spaced locations along the stop locator to allow one to properly position stops along the length of the needle. Those stops aid in the desired placement of the needle at a specific location in a manner described elsewhere in this document.

In one or more of the many possible embodiments of the apparatus, the apparatus further includes a support adapted for mounting the needle guide to a probe, such as an ultrasound probe. The support may include a receiver for receiving and holding the needle guide. That receiver may comprise a spring clip that allows the needle guide to snap into place on the support. That spring clip may further include a release lever so that one may remove the needle guide from the support when desired.

In one or more of the many possible embodiments of the apparatus, the apparatus includes a first additional needle guide. Further, the apparatus may also include a second additional needle guide. In such embodiments, the plurality of apertures in the needle guide may be oriented at an angle A)(e.g. 30° relative to the longitudinal axis of the needle guide. The plurality of apertures in the first additional needle guide may be oriented at an angle B (e.g. 45°) relative to a longitudinal axis of the first additional needle guide. The plurality of apertures in the second additional needle guide may be oriented at an angle C)(e.g. 60° relative to a longitudinal axis of the second additional needle guide. Further, the needle guide, the first additional needle guide and the second additional needle guide may be interchangeably connected to the receiver of the support as needed in order to properly place the needle at a desired position under the skin of the patient in a manner set forth below.

In the following description, there are shown and described several preferred embodiments of the apparatus. As it should be realized, the apparatus is capable of other, different embodiments and its several details are capable of modification in various, obvious aspects all without departing from the apparatus as set forth and described in the following claims. Accordingly, the drawings and descriptions should be regarded as illustrative in nature and not as restrictive.

### BRIEF DESCRIPTION OF THE DRAWING FIGURES

The accompanying drawing figures incorporated herein and forming a part of the specification, illustrate several aspects of the apparatus for placing a needle at a specific location and depth using an probe and together with the description serve to explain certain principles thereof.

FIG. 1 is a perspective view of the components of the apparatus including (a) the assembled needle guide, (b) the assembled support, (c) the stop locator, (d) the syringe, (e) the needle with three stops held on the needle and (f) the probe.

FIG. 2 is a detailed perspective view of the two sections of the needle guide.

FIG. 3 is a detailed perspective view of the probe positioned between the two portions of the support.

FIG. 4A is a detailed perspective view of the stop locator.

FIG. 4B is a view similar to FIG. 4A but illustrating how the stop locator is used to position stops at desired positions along the shaft of the needle to allow one to confidently control the depth of the needle tip under the skin during a medical procedure.

FIG. 5A is a detailed perspective view illustrating how the mounting end of the needle guide is inserted in the receiver of the support.

FIG. 5B is a detailed perspective view illustrating how the needle is inserted into one of the needle guide apertures in order to ultimately place the needle in the desired location under the skin of a patient.

FIGS. 5C-5E are schematic side elevational views illustrating how one uses the apparatus to position the needle within a vessel under the skin of the patient.

FIG. 5F is a schematic side elevational view showing how the needle guide is released from the support with the release lever, the probe is removed and the needle guide separates from around the needle.

FIG. 6 is a partial cross-section of three needle guides wherein each includes needle guide apertures at different angles.

FIG. 7 is a top plan view illustrating an alternative embodiment of the needle guide including three parallel rows of needle guide apertures extending along the length of the needle guide.

FIG. 8 is a side elevational view of an alternative embodiment of the needle guide incorporating the stop locator.

FIG. 9 is a top plan view of an alternative embodiment of the apparatus including two receivers for the needle guide wherein the receivers are located along axes separated by 90°.

### DETAILED DESCRIPTION

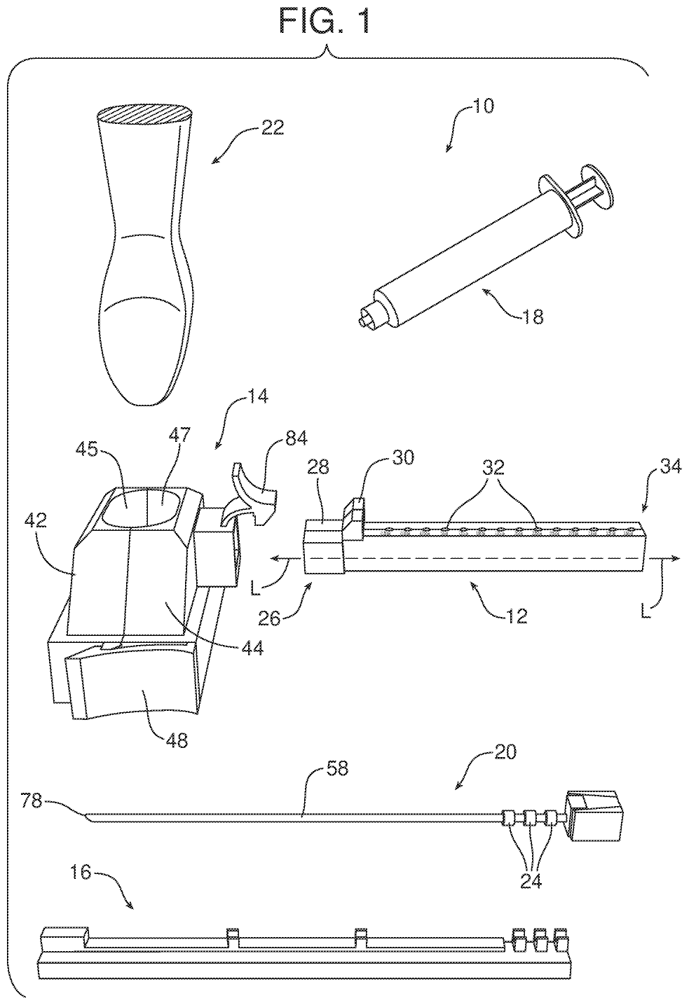

Reference is now made to FIGS. 1-5F which illustrate a first possible embodiment of the apparatus 10. As illustrated in FIG. 1, the apparatus 10 may be considered to include a number of different components: (a) a needle guide 12 (shown assembled in FIG. 1), (b) a support 14 (also shown assembled in FIG. 1), (c) a stop locator 16, (d) a syringe 18, (e) a needle 20 and (f) a probe 22, such as an ultrasonic probe of a type known in the art. In FIG. 1, the needle 20 includes three stops 24 along the shaft 58 that function in a manner that will become clear in the following description.

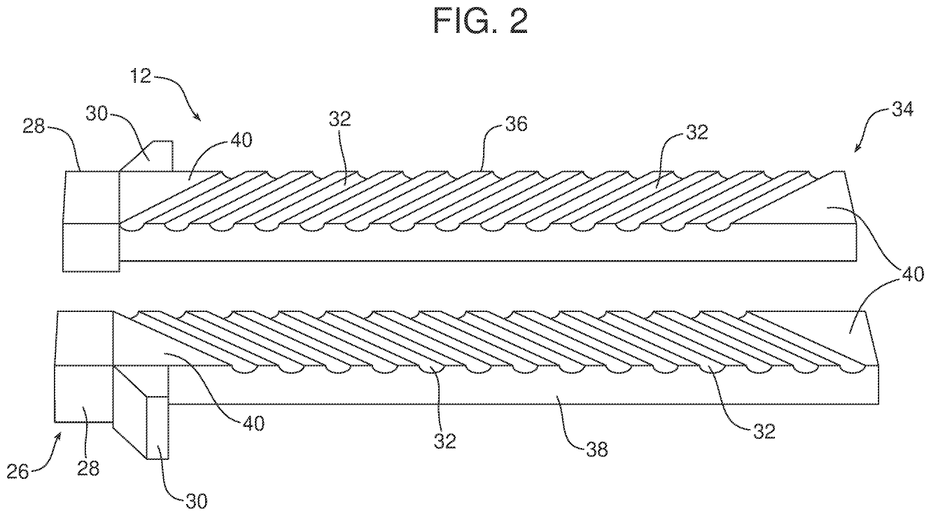

As illustrated in FIGS. 1 and 2, the needle guide 12 includes a mounting end 26 including a terminal projection 28 adjacent a locking lug 30. A plurality of needle guide apertures 32 extend through the needle guide 12. In the embodiment illustrated in FIGS. 1 and 2, the plurality of needle guide apertures 32 are oriented in a single row extending along the length of the needle guide 12 from the locking lug 30 to the distal end 34 of the needle guide opposite the mounting or proximal end 26. The needle guide apertures 32 may be oriented at an angle of between about 1°-89° (more commonly between about 30°-60° relative to a longitudinal axis L of the needle guide 12.

The needle guide 12 may be made from any metal, plastic or composite material suitable for such a purpose. The needle guide 12 may be a single piece construction. Alternatively, as illustrated in FIG. 2, the needle guide may be made from two sections 36, 38 that are substantial mirror images of each other. The two sections 36, 38 may be interconnected along an interface 40 that extends along the longitudinal axis L of the needle guide and through each needle guide aperture 32.

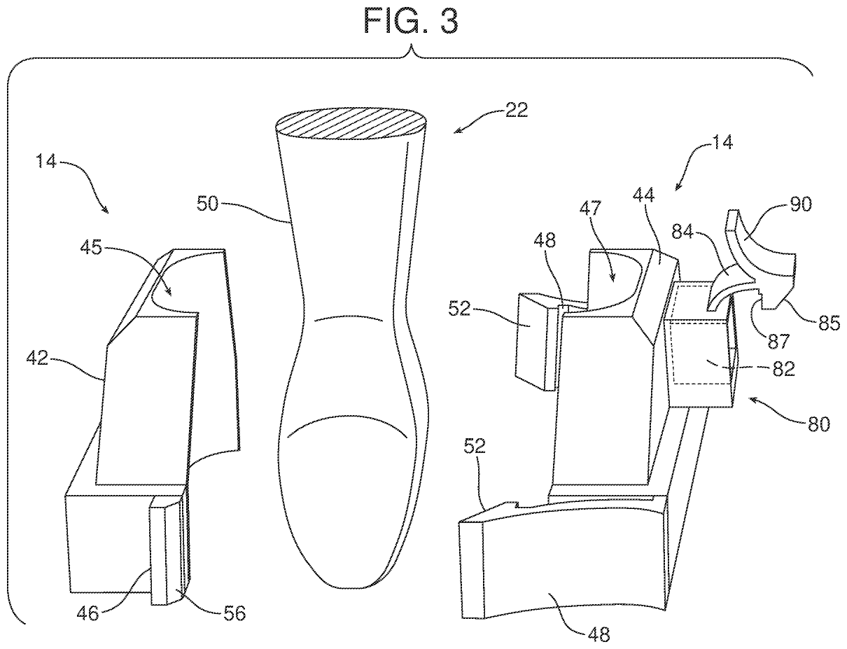

As illustrated in FIGS. 1 and 3, the support 14 comprises a two piece body 42, 44. Body piece 42 includes a cavity 45 and two locking lugs 46 while body piece 44 includes a cavity 47 and two resilient locking tabs 48. The support 14 is secured tightly around the outer housing 50 of the probe 22 by (a) aligning the support with respect to the probe so that the probe fits in the cavities 45, 47 and (b) securing the two body pieces 42, 44 together around the probe to provide a friction fit. When properly connected, the two locking tabs 48 on the body piece 44 engage and lock to the two cooperating locking lugs 46 on the body piece 42. More particularly, the two locking ribs 52 carried on the locking tabs 48 snap over the locking lugs 46 to complete the connection (note particularly, the beveled edges 54 on the locking ribs 52 and the cooperating beveled edges 56 on the locking lugs 46 that aid in making this connection).

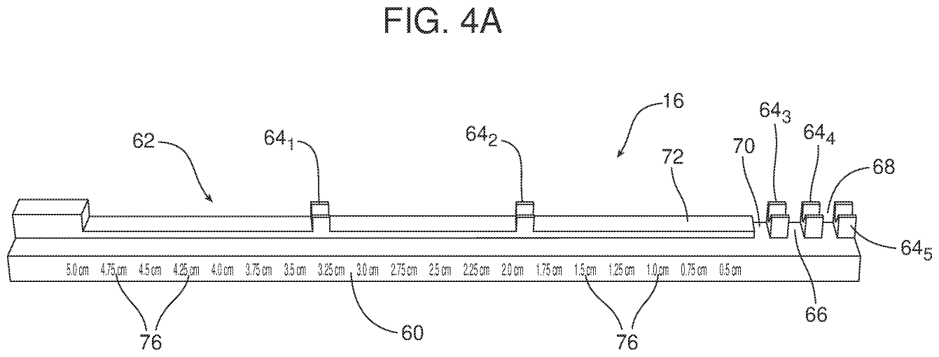

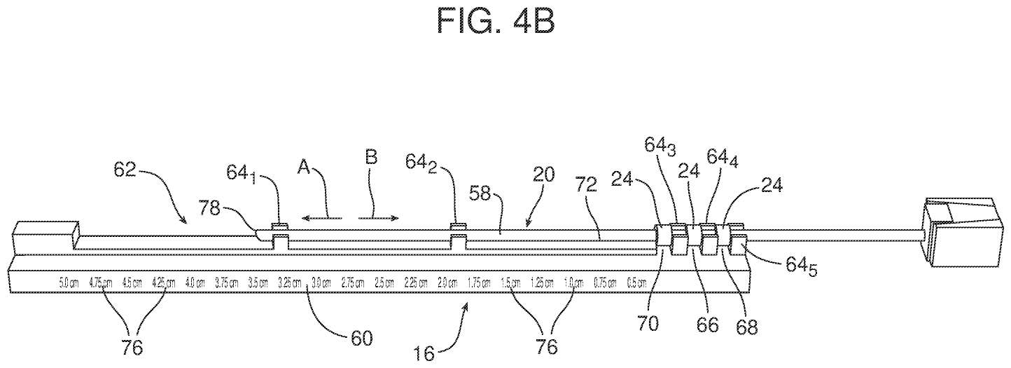

As illustrated in FIGS. 1 and 4, the stop locator 16 is adapted for positioning a plurality of stops 24 along the shaft 58 of the needle 20. These stops 24 aid in the proper positioning of the needle 20 under the skin of the patient in a manner described elsewhere in this document. Each stop 24 may be made from rubber or other appropriate resilient material that resists sliding along the shaft 58 of the needle. In one possible embodiment, each stop 24 may have a length of about 3 mm. The stops may also be spaced apart by a distance of, for example, about 3 mm.

The stop locator 16 includes a body 60 including a needle track 62 defined by a plurality of spaced resilient clips 64₁₋₅ that are adapted to receive and hold the needle 20. Space 66 provided between the clips 64₃ and 64₄, space 68 provided between the clips 64₄ and 64₅ and space 70 provided between the clip 64₃ and the raised rib 72 formed in the body 60 of the stop locator 16 are adapted to receive and position the stops 24 at the desired locations along the shaft 58 of the needle 20. For purposes of this document, those spaces 64, 66 and 68 are referred to as stop receivers. A series of graduations 76 are marked on the body 60 along the needle track 62. The graduations represent different depths for needle penetration under the skin.

In use, the three stops 24 are positioned over the shaft 58 of the needle 20. The needle 20 is then inserted into the needle track 62 by pressing the needle down into the spaced resilient clips 64₁₋₅ while also pushing the stops 24 along the shaft 58 so that the three stops are received in their own individual stop receiver 66, 68, 70. See FIG. 4B. The needle 20 is then shifted in the direction of the action arrows A and/or B with respect to the stop locator 16 along the needle track 62 until the tip 78 of the needle is aligned with the particular graduation 76 on the body 72 indicating the desired depth of needle penetration under the skin of a patient. While the needle 20 is being shifted, the stops 24 are held in the stop receivers 66, 68 and 70 and properly positioned along the shaft 58 of the needle for positioning of the needle at the desired depth under the skin. One then carefully snaps the needle 20 out of the clips with the stops 24 in their proper place. Here it should be noted that the positioning of the stops 24 at the desired locations along the shaft 58 of the needle 20, as described and shown, may be performed with or without the needle being connected to the syringe 18.

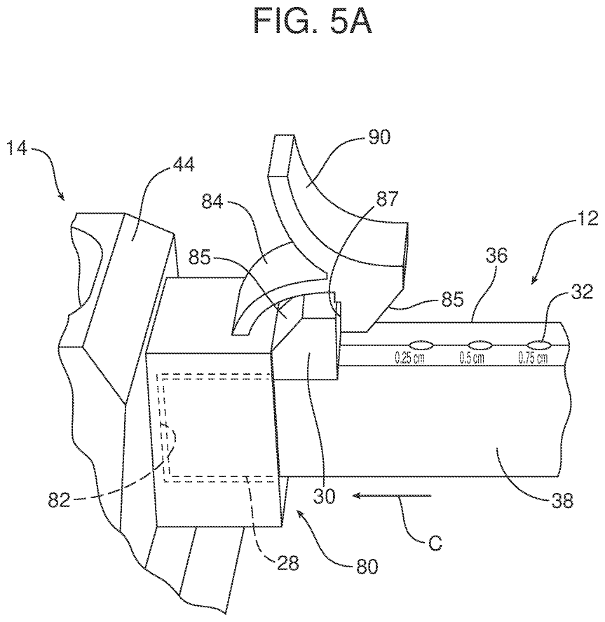

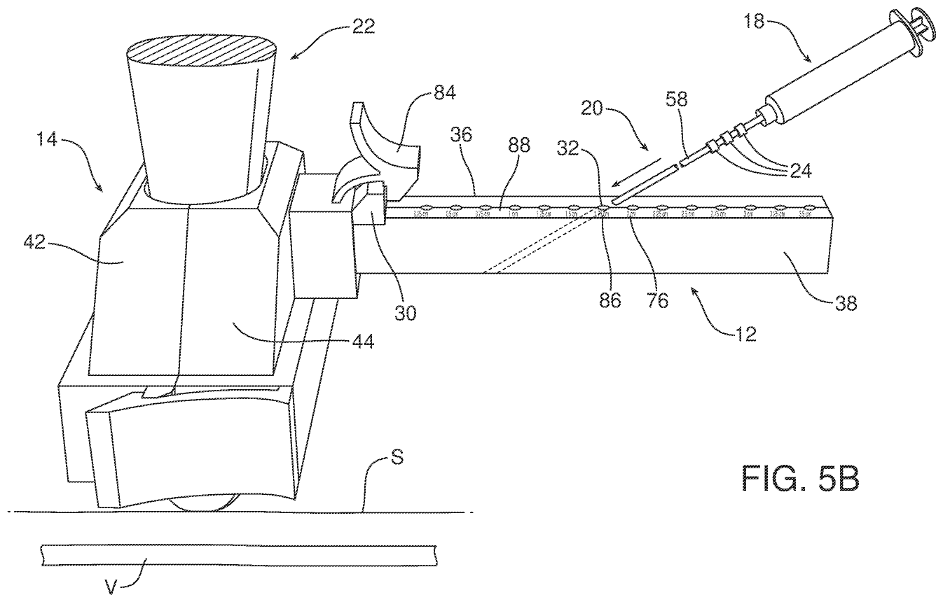

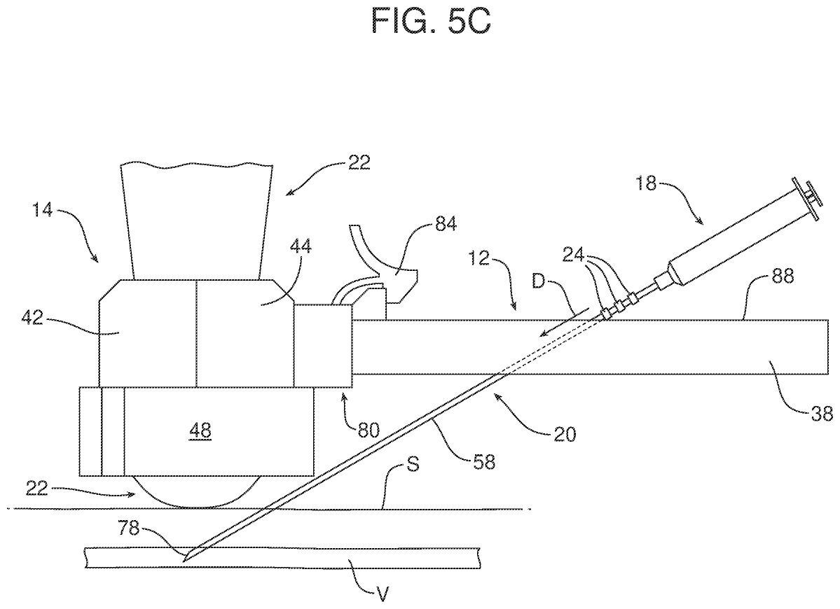

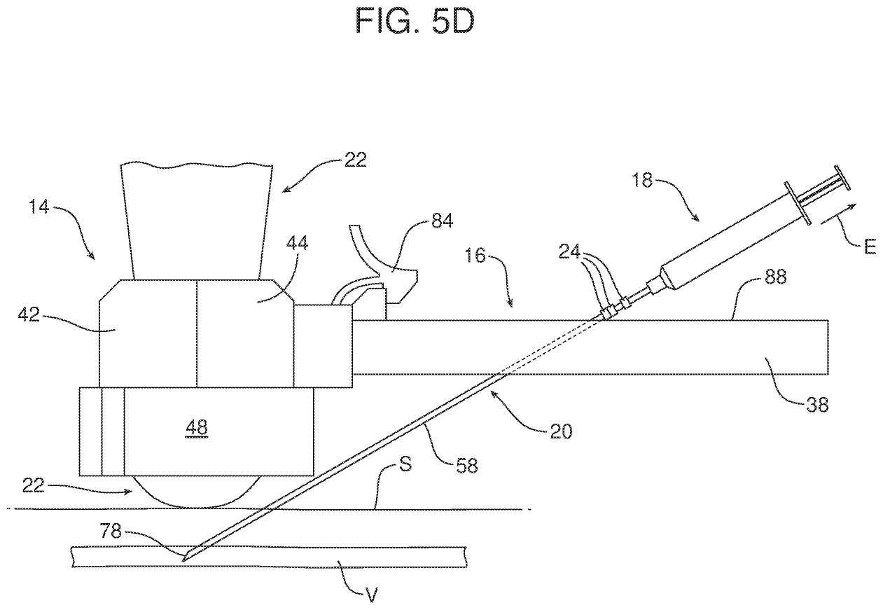

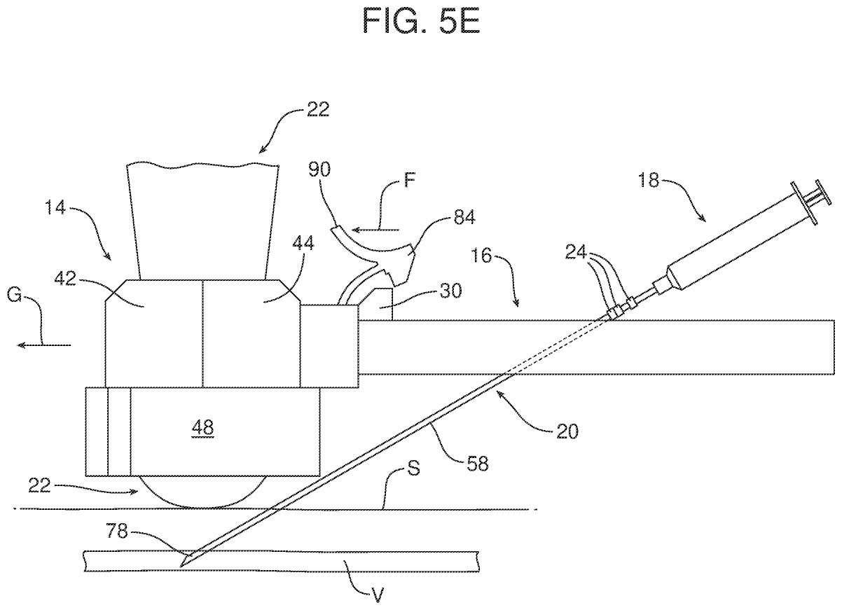

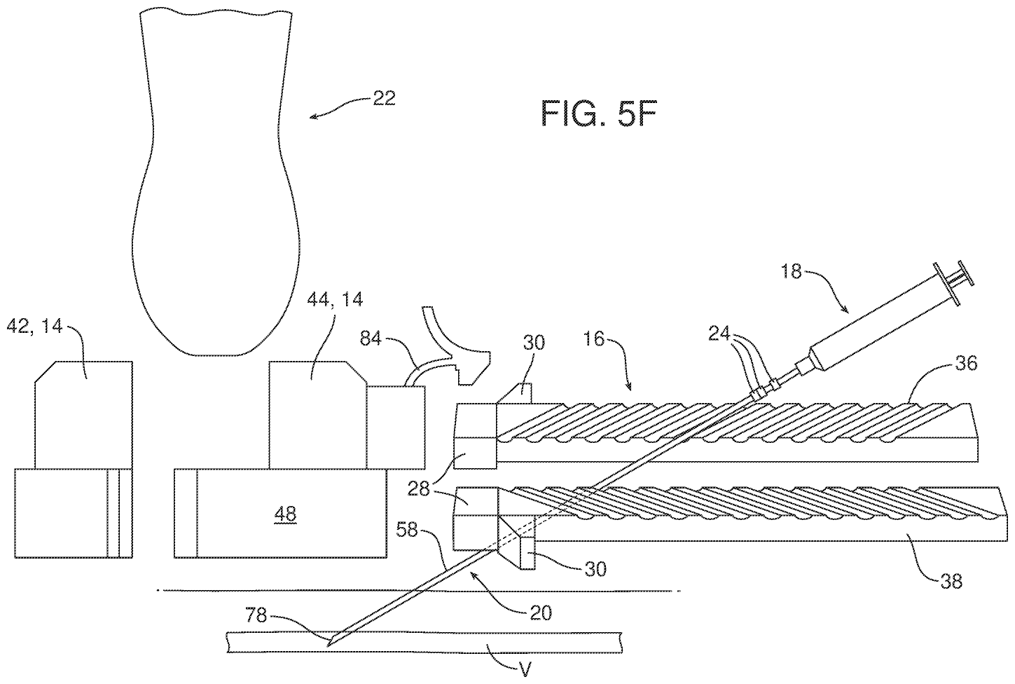

Reference is now made to FIGS. 5A-5F which serve to illustrate the use of the apparatus 10 for purposes of the Seldinger Technique. For purposes of this explanation, the support 14 has been previously attached to the probe 22 in the manner described above and illustrated in FIG. 3. The probe 22 is then used, in a manner known in the art, to determine the desired depth for the injection to be administered using the syringe 22 and the needle 20. For purposes of this explanation, let us assume that the probe 22 identifies the vessel V for injection with the center of the vessel located at 1.75 cm under the skin at the injection site.

The stop locator 16 is then used in the manner described above and illustrated in FIG. 4 to position the stops 24 along the shaft 58 of the needle 20 for penetration at the desired depth of 1.75 cm by shifting the tip 78 of the needle along the needle track 62 until it is aligned with the 1.75 cm graduation 76. The needle 20 is then carefully removed from the stop locator 16 and attached to the syringe 22, if not already so attached.

If not already done, the needle guide 12 is attached to the support 14 secured to the probe 22. As illustrated in FIG. 5A, this may be done by inserting (note action arrow C) the terminal projection 28 at the mounting end of the needle guide 12 into the receiver 80 carried on the body piece 44 of the support 14. More particularly, that receiver 80 includes a socket 82 and a spring clip 84. The terminal projection 28 is aligned with and inserted into the socket 82 to produce a snug friction fit. As the terminal projection 28 is inserted into the socket 82, the locking lug 30 is engaged by the spring clip 84 to secure the needle guide 12 in proper position on the support 14. Note cooperating beveled surfaces 85 on the spring clip 84 and the lug 30 that aid in making this connection. When fully inserted, the shoulder 87 on the spring clip 84 engages the lug 30 to complete a secure connection.

With the probe 22 positioned against the skin S over the injection site, the tip 78 of the needle 20 is inserted into the needle guide aperture 32 associated with the graduation 86 on the top face 88 of the needle guide indicating the desired penetration depth of 1.75 cm corresponding to the depth at the center of the target vessel V. See FIG. 5B.

The needle 20 is then pushed down (note action arrow D) through the selected needle guide aperture 32 until the first stop 24 engages the top face 88 of the needle guide 12, at which time the tip 78 of the needle 20 may be visualized with the probe 22 at the desired depth at the injection site with the tip located in the center of the vessel V. See FIG. 5C. When the first stop 24 engages the top face 88 of the needle guide 12, a resistance is provided to further penetration of the needle 20 under the skin S (i.e. the stop resists sliding along the shaft 58 of the needle giving the user of the apparatus 10 a clear tactile indication that the tip 78 of the needle is at the desired depth as may be confirmed on the probe monitor (not shown)).

If the vessel V collapses or tissue movement causes the target to be slightly further than expected, a quick “pop” of the needle 20 may still be required to penetrate the outer wall of the vessel V. This can be done with confidence as any further needle penetration resulting from the “pop” will be limited by the second stop 24. This is because, when the second stop 24 comes into engagement with the first stop 24 at the top face 88 of the needle guide 16, additional resistance to further needle penetration is provided (note FIG. 5D). Once the needle tip 78 is in the vessel V as confirmed by the stops 24 and the view on the probe monitor (not shown), one manipulates the plunger 79 of the syringe 18 (note action arrow E) to draw negative pressure. If the tip 78 of the needle 20 is properly located in the vessel V, this will serve to draw blood into the syringe 18, thereby confirming proper needle placement. If, for some reason still deeper needle penetration is desired, the third stop 24 will engage the other two stops providing still more resistance to needle penetration thereby protecting body structures from over penetration of the needle.

As illustrated in FIG. 5E, in order to release the needle guide 12 from the needle 20, one depresses the release lever 90 on the spring clip 84 to release the locking lug 30 (see action arrow F). Next one shifts the probe 22 in the direction of action arrow G to free the terminal projection 28 at the mounting end of the needle guide 12 from the socket 84. As a result, the two sections 36, 38 of the needle guide separate along the interface 40, freeing the needle 20 from the needle guide aperture 32. See FIG. 5F.

The syringe 18 is removed from the needle 20 in a manner known in the art. A guidewire (not shown) may then be advanced through the needle 20 and into the vessel V in a manner known in the art. Once this guidewire is in place, the needle 20 is removed off of the wire, and a catheter, dilator or sheath, of a type known in the art and not shown, is then advanced over the guidewire into the vessel. The guidewire is then removed and the desired catheter, dilator or sheath remains in the vessel V, so other procedures, such as blood draw, medication administration, or endovascular procedures, may be carried out.

While the use described above relates to the Seldinger Technique, it should be appreciated that the apparatus 10 may be used for other applications such as simple injection, biopsy, drainage of an abscess cavity or various other medical uses associated with syringes and needles.

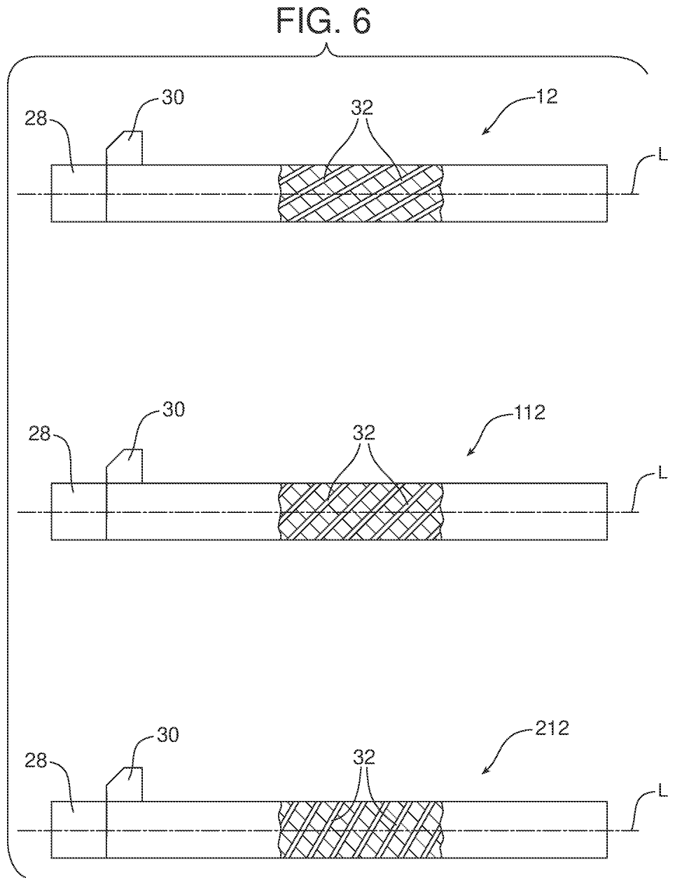

Reference is now made to FIG. 6, illustrating that in at least one embodiment of the apparatus 10, the apparatus may include three separate needle guides 12, 112 and 212. The three needle guides are identical in all respects except for the angle of the plurality of needle guide apertures. Thus, each needle guide 12, 112, 212 includes a mounting end having a terminal projection 28 and a locking lug 30 allowing the needle guides 12, 112, 212 to be interchangeably connected to the support 14 by means of the receiver 80 in the manner described above.

The plurality of needle guide apertures 32 in the first needle guide 12 are all parallel at an angle A (e.g. 30°) relative to the longitudinal axis L of the needle guide 12. In contrast, the plurality of needle guide apertures 32 in the first additional needle guide 112 are all parallel at an angle B (e.g. 45° relative to the longitudinal axis L of the needle guide 112. In still further contrast, the plurality of needle guide apertures 32 in the second additional needle guide 212 are all parallel at an angle C (e.g. 60°) relative to the longitudinal axis L of the needle guide 212. In other words, the angles A, B and C are all different.

One may select the needle guide 12, 112, 212 providing the desired needle penetration angle for a particular procedure and attached that needle guide to the support 14 on the probe 22. One then inserts the tip 78 of the needle 20 into the particular needle guide aperture 32 associated with the graduation 86 corresponding to the desired penetration depth indicated by the probe. The depth indicated by the graduations 86 on the needle guides 12, 112, 212 for each needle guide aperture adjusts for the penetration angle of the needle guide apertures 32 to ensure that the tip 78 of the needle 20 is at the desired depth when it is under the probe 22.

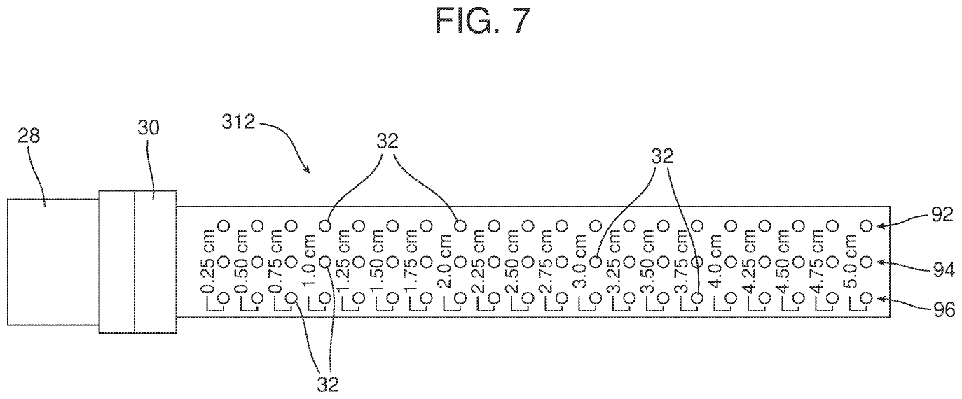

Reference is now made to FIG. 7 illustrating yet another alternative embodiment of the needle guide 12. This alternative embodiment of the needle guide 312 includes three parallel rows 92, 94, 96 of needle guide apertures 32. In one possible embodiment, all of the needle guide apertures 32 in the three rows 92, 94, 96 are oriented at the same penetration angle of between about 30°-60°. This allows the user to shift between the rows of needle guide apertures 32 to adjust the position of the tip 78 of the needle laterally if needed.

In other possible embodiments, the needle guide apertures 32 in each row 92, 94, 96 are oriented at different penetration angles. For example, the needle guide apertures 32 in the first row 92 may be oriented at a penetration angle of about 30°, the needle guide apertures in the second row 94 may be oriented at a penetration angle of about 45° and, the needle guide apertures in the third row 96 may be oriented at a penetration angle of about 60°. This allows the user to change needle penetration angles if desired for any reason.

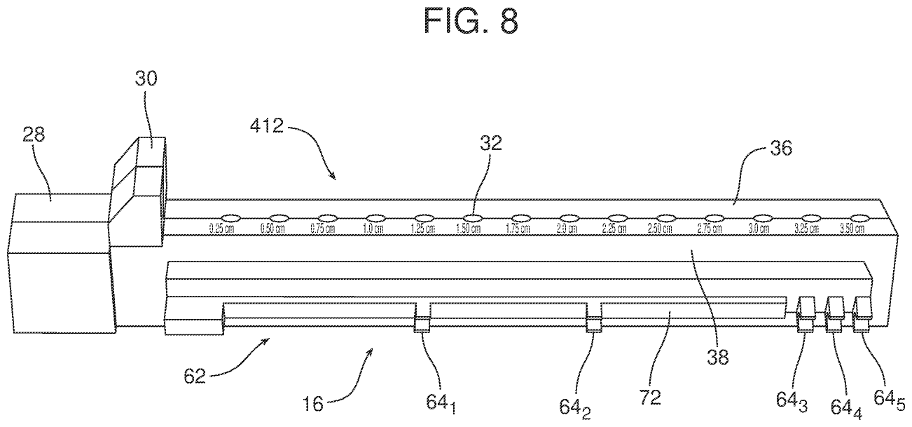

Reference is now made to FIG. 8 illustrating yet another alternative embodiment of the needle guide wherein the needle guide 412 incorporates or includes the stop locator 16. Many users would find this arrangement more convenient to use. This embodiment also consists of one less part.

The embodiment, illustrated in FIGS. 1, 3 and 5B-5E, relates to an embodiment wherein the support 14 includes a single receiver 80 and socket 82 at the front face or a long side of the probe 22. It should be appreciated that the receiver 80 could be provided along any other face of the probe 22, including the back or opposite long side and either end or short side of the probe. In the embodiment illustrated in FIGS. 1 and 3, the locking lugs 46 and the resilient locking tabs 48 are provided on the body pieces 42, 44 at opposed ends or short sides of the probe 22. Alternatively, they could be provided on the opposed long sides of the probe 22 if desired.

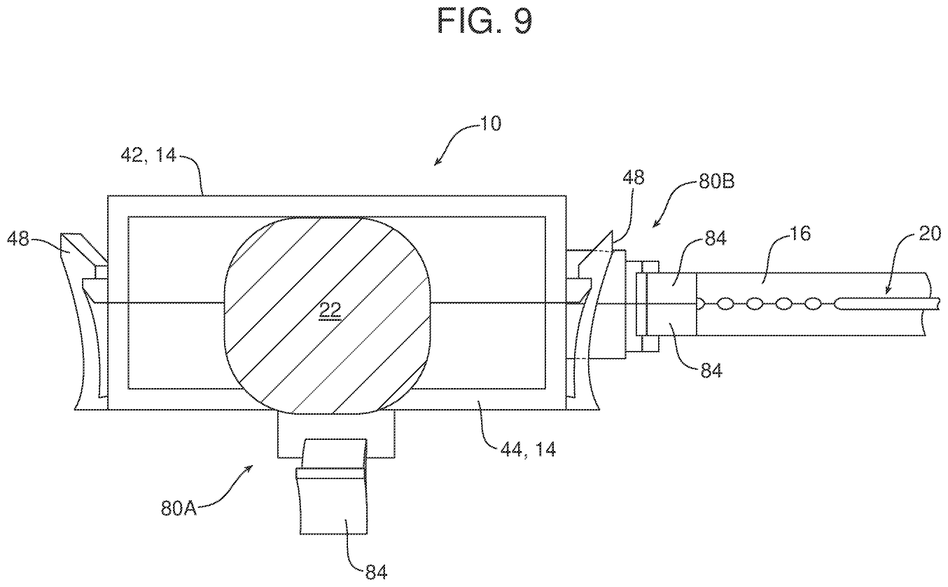

In an alternative embodiment of the apparatus 10 illustrated in FIG. 9, two receivers 80A, 80B are provided. Receiver 80A is oriented along the front or one of the long sides of the probe 22 while receiver 80B is oriented along one of the ends or short sides of the probe. A user may attach the needle guide 12 in either receiver 80A or 80B depending upon the monitor view desired by the user. More specifically, when the needle guide 12 is mounted in a receiver at an end or short side of the probe 22, one can obtain a longitudinal view with the probe 22 as the needle 20 is used to penetrate a vessel V. Such a view is often preferred as it allows for viewing of the entire length and trajectory of the needle during the insertion procedure.

This disclosure may be said to relate to the following items.
1. An apparatus, comprising:
   a needle guide including a plurality of needle guide apertures.
2. The apparatus of item 1, wherein the needle guide apertures are oriented in a first row extending along a length of the needle guide.
3. The apparatus of item 2, wherein the needle guide apertures are also oriented in a second row extending parallel to the first row.
4. The apparatus of item 3, wherein the needle guide apertures are also oriented in a third row extending parallel to the first row and the second row.
5. The apparatus of item 4, wherein said needle guide apertures are oriented at an angle of between about 1°-89° relative to a longitudinal axis of the needle guide.
6. The apparatus of any of items 1-5, further including needle depth graduations along a face of the needle guide.
7. The apparatus of item 6, further including a stop locator adapted for positioning a plurality of stops on a needle to be guided by the needle guide.
8. The apparatus of item 7, wherein the stop locator includes depth graduations and a plurality of stop receivers at spaced locations along the stop locator.
9. The apparatus of item 8, further including a support adapted for mounting the needle guide to a probe.
10. The apparatus of item 9, wherein the support includes a receiver for receiving and holding the needle guide.
11. The apparatus of item 10, wherein the receiver comprises a spring clip.
12. The apparatus of item 11, wherein the spring clip further includes a release lever.
13. The apparatus of item 12, further including a first additional needle guide.
14. The apparatus of item 13, further including a second additional needle guide wherein (a) the plurality of apertures in needle guide are oriented at an angle A relative to the longitudinal axis of the needle guide, (b) a plurality of apertures in the first additional needle guide are oriented at an angle B relative to a longitudinal axis of the first additional needle guide, (c) a plurality of apertures in the second additional needle guide are oriented at an angle of C relative to a longitudinal axis of the second additional needle guide, where angles A, B and C are all different, and (d) the needle guide, the first additional needle guide and the second additional needle guide may be interchangeably connected to the receiver.
15. The apparatus of item 1, further including a support adapted for mounting the needle guide to a probe wherein the support includes a receiver.
16. The apparatus of item 15, further including a first additional needle guide and a second additional needle guide wherein (a) the plurality of apertures in the needle guide are oriented at an angle A relative to the longitudinal axis of the needle guide, (b) a plurality of apertures in the first additional needle guide are oriented at an angle B relative to a longitudinal axis of the first additional needle guide, (c) a plurality of apertures in the second additional needle guide are oriented at an angle C relative to a longitudinal axis of the second additional needle guide, where angles A, B and C are all different, and (d) the needle guide, the first additional needle guide and the second additional needle guide may be interchangeably connected to the receiver.
17. The apparatus of item 8, further including an ultrasonic probe and a support adapted for mounting the needle guide to the ultrasonic probe.
18. The apparatus of item 17, further including a syringe wherein the needle carrying the plurality of stops is attached to the syringe and the needle is positioned in one of the plurality of apertures in the needle guide.
19. The apparatus of item 1, wherein the needle guide includes a first section and a second section interconnected along an interface extending along a longitudinal axis of the needle guide and through each aperture of the plurality of needle guide apertures.
20. The apparatus of item 1, further including a support adapted for mounting the needle guide to a probe.
21. The apparatus of item 20, wherein the support includes a receiver for receiving and holding the needle guide.
22. The apparatus of item 21, wherein the receiver includes a socket and a cooperating spring clip.
23. The apparatus of item 22, wherein the needle guide includes a mounting end having a terminal projection adjacent a locking lug and when the needle guide is secured to the support, the terminal projection is engaged in the socket and the spring clip is engaged with the locking lug.
24. The apparatus of item 1, wherein the needle guide includes a mounting end having a terminal projection adjacent a locking lug.
25. The apparatus of item 20, wherein the support includes a two piece body.
26. The apparatus of item 25, wherein a first piece of the two piece body includes a first cavity and two locking lugs.
27. The apparatus of item 26, wherein a second piece of the two piece body includes a second cavity and two resilient locking tabs wherein when the support is secured to the probe, the probe is received in the first and second cavities and the two locking tabs on the second piece engage and lock to the two cooperating locking lugs on the first piece.
28. The apparatus of item 8, wherein the stop locator further includes a needle track defined by a plurality of spaced resilient clips that are adapted to receive and hold the needle while the stops are being located in desired positions of the needle.
29. The apparatus of item 8, wherein the stop locator is carried on the needle guide.
30. The apparatus of item 1, further including a support adapted for mounting the needle guide to a probe wherein the support includes a first receiver and a second receiver wherein the needle guide is received and held in either of the first receiver or the second receiver.

Each of the following terms written in singular grammatical form: “a”, “an”, and “the”, as used herein, means “at least one”, or “one or more”. Use of the phrase “one or more” herein does not alter this intended meaning of “a”, “an”, or “the”. Accordingly, the terms “a”, “an”, and “the”, as used herein, may also refer to, and encompass, a plurality of the stated entity or object, unless otherwise specifically defined or stated herein, or, unless the context clearly dictates otherwise. For example, the phrase: “a needle guide”, as used herein, may also refer to, and encompass, a plurality of needle guides.

Each of the following terms: “includes”, “including”, “has”, “having”, “comprises”, and “comprising”, and, their linguistic/grammatical variants, derivatives, or/and conjugates, as used herein, means “including, but not limited to”, and is to be taken as specifying the stated component(s), feature(s), characteristic(s), parameter(s), integer(s), or step(s), and does not preclude addition of one or more additional component(s), feature(s), characteristic(s), parameter(s), integer(s), step(s), or groups thereof.

The phrase “consisting of”, as used herein, is closed-ended and excludes any element, step, or ingredient not specifically mentioned. The phrase “consisting essentially of”, as used herein, is a semi-closed term indicating that an item is limited to the components specified and those that do not materially affect the basic and novel characteristic(s) of what is specified.

Terms of approximation, such as the terms about, substantially, approximately, etc., as used herein, refers to ±10% of the stated numerical value. It is to be fully understood that certain aspects, characteristics, and features, of the apparatus, which are, for clarity, illustratively described and presented in the context or format of a plurality of separate embodiments, may also be illustratively described and presented in any suitable combination or sub-combination in the context or format of a single embodiment. Conversely, various aspects, characteristics, and features, of the apparatus which are illustratively described and presented in combination or sub-combination in the context or format of a single embodiment may also be illustratively described and presented in the context or format of a plurality of separate embodiments.

Although the apparatus has been illustratively described and presented by way of specific exemplary embodiments, and examples thereof, it is evident that many alternatives, modifications, or/and variations, thereof, will be apparent to those skilled in the art. For example, a needle guide 12 with a single row of needle guide apertures 32 is illustrated in FIGS. 1 and 2 and a needle guide 312 with three rows of needle guide apertures 32 is illustrated in FIG. 7. Needle guides with other numbers of needle guide aperture rows are possible. Needle guide apertures 32 at penetration angles of 30°, 45° and 60° are described above and illustrated in the drawing figures. Other penetration angles are possible.

It is intended that all such alternatives, modifications, or/and variations, fall within the spirit of, and are encompassed by the broad scope of the appended claims.

1. An apparatus, comprising:
a needle guide including a plurality of needle guide apertures and a stop locator adapted for positioning a plurality of stops on a needle to be guided by the needle guide.

2. (canceled)

3. (canceled)

4. (canceled)

5. (canceled)

6. (canceled)

7. (canceled)

8. The apparatus of claim 1, wherein the stop locator includes depth graduations and a plurality of stop receivers at spaced locations along the stop locator.

9. The apparatus of claim 8, further including a support adapted for mounting the needle guide to a probe.

10. The apparatus of claim 9, wherein the support includes a receiver for receiving and holding the needle guide.

11. The apparatus of claim 10, wherein the receiver comprises a spring clip.

12. The apparatus of claim 11, wherein the spring clip further includes a release lever.

13. The apparatus of claim 12, further including a first additional needle guide.

14. The apparatus of claim 13, further including a second additional needle guide wherein (a) the plurality of apertures in the needle guide are oriented at an angle A relative to the longitudinal axis of the needle guide, (b) a plurality of apertures in the first additional needle guide are oriented at an angle B relative to a longitudinal axis of the first additional needle guide, (c) a plurality of apertures in the second additional needle guide are oriented at an angle C relative to a longitudinal axis of the second additional needle guide, where angles A, B and C are all different, and (d) the needle guide, the first additional needle guide and the second additional needle guide may be interchangeably connected to the receiver.

15. The apparatus of claim 1, further including a support adapted for mounting the needle guide to a probe wherein the support includes a receiver.

16. The apparatus of claim 15, further including a first additional needle guide and a second additional needle guide wherein (a) the plurality of apertures in the needle guide are oriented at an angle A relative to the longitudinal axis of the needle guide, (b) a plurality of apertures in the first additional needle guide are oriented at an angle B relative to a longitudinal axis of the first additional needle guide, (c) a plurality of apertures in the second additional needle guide are oriented at an angle C relative to a longitudinal axis of the second additional needle guide, where angles A, B and C are all different, and (d) the needle guide, the first additional needle guide and the second additional needle guide may be interchangeably connected to the receiver.

17. The apparatus of claim 8, further including an ultrasonic probe and a support adapted for mounting the needle guide to the ultrasonic probe.

18. The apparatus of claim 17, further including a syringe wherein the needle carrying the plurality of stops is attached to the syringe and the needle is positioned in one of the plurality of apertures in the needle guide.

19. The apparatus of claim 1, wherein the needle guide includes a first section and a second section interconnected along an interface extending along a longitudinal axis of the needle guide and through each aperture of the plurality of needle guide apertures.

20. (canceled)

21. (canceled)

22. (canceled)

23. The apparatus of claim 1, further including a support adapted for mounting the needle guide to a probe wherein (a) the support includes a receiver for receiving and holding the needle guide, (b) the receiver includes a socket and a cooperating spring clip and (c) the needle guide includes a mounting end having a terminal projection adjacent a locking lug and when the needle guide is secured to the support, the terminal projection is engaged in the socket and the spring clip is engaged with the locking lug.

24. An apparatus comprising, a needle guide including a plurality of needle apertures wherein the needle guide includes a mounting end having a terminal projection adjacent a locking lug.

25. An apparatus, comprising, a needle guide including a plurality of needle apertures and a support adapted for mounting the needle guide to a probe wherein the support includes a two piece body.

26. The apparatus of claim 25, wherein a first piece of the two piece body includes a first cavity and two locking lugs.

27. The apparatus of claim 26, wherein a second piece of the two piece body includes a second cavity and two resilient locking tabs wherein when the support is secured to the probe, the probe is received in the first and second cavities and the two locking tabs on the second piece engage and lock to the two cooperating locking lugs on the first piece.

28. The apparatus of claim 8, wherein the stop locator further includes a needle track defined by a plurality of spaced resilient clips that are adapted to receive and hold the needle while the stops are being located in desired positions of the needle.

29. The apparatus of claim 8, wherein the stop locator is carried on the needle guide.

30. (canceled)
